(19) United States
(12) Patent Application Publication
(10) Pub. No.: US 2016/0084851 A1
(43) Pub. Date: Mar. 24, 2016

(54) NOVEL BIOMARKERS

(71) Applicant: ProteinLogic Limited, Cambridge (GB)

(72) Inventors: Graham Foster, Cambridge (GB); Paul Rodgers, Cambridge (GB); Oliver Stegle, Cambridge (GB)

(73) Assignee: ProteinLogic Limited, Cambridge (GB)

(21) Appl. No.: 14/786,173

(22) PCT Filed: Apr. 23, 2014
(86) PCT No.: PCT/GB2014/051253

§ 371 Date: Oct. 22, 2015

(30) Foreign Application Priority Data
Apr. 23, 2013 (GB) 1307267.3

(51) Int. Cl. G01N 33/74

(52) U.S. Cl. CPC G01N 33/74; G01N 2800/52; G01N 2333/72

(57) ABSTRACT

The invention relates to biomarkers for predicting whether a patient infected with Hepatitis B virus (HBV) will respond to interferon (IFN) therapy. The invention also relates to methods and kits for predicting whether a patient will respond to IFN therapy using said biomarkers.

### FIELD OF THE INVENTION

The invention relates to biomarkers for predicting whether a patient infected with Hepatitis B virus (HBV) will respond to interferon (IFN) therapy. The invention also relates to methods and kits for predicting whether a patient will respond to IFN therapy using said biomarkers.

### BACKGROUND OF THE INVENTION

Hepatitis B is a disease caused by the Hepatitis B virus (HBV). HBV mainly infects cells in the liver known as hepatocytes and results in liver damage as the host immune response tries to clear the viral infection.

Despite an effective vaccination and post-exposure prophylaxis being available, Hepatitis B virus (HBV) infection is still prevalent worldwide and is a serious global health problem accounting for significant morbidity and mortality. More than 2 billion people alive today have been infected with HBV at some time in their lives. Three quarters of the world's population live in areas where there are high levels of infection. Every year there are over 4 million acute clinical cases of HBV.

Although a majority of patients recover from the acute infection, approximately 350 million people worldwide (5% of world population) suffer from chronic HBV infection. This is the area where a lot of progress has been made over the years to understand the clinical course of the disease and several therapies have been approved worldwide. The effectiveness of current therapies is not 100% and there is much on-going research into novel therapies.

Chronic HBV infection is the 10th leading cause of death worldwide, resulting in 1 to 1.2 million deaths per year. Approximately 15-40% of chronically infected patients will develop cirrhosis, liver failure, or hepatocellular carcinoma (HCC). HCC incidence has increased worldwide, and it is now the 5th most frequent cancer, accounting for 300,000 to 500,000 diagnosed cases each year.

There are 8 viral genotypes of HBV (A-H) and these vary in geographic distribution (see Table 1). Several genotypes may be associated with the severity of the disease but the relationship between the genotype and risk of HCC has not yet been established.

TABLE 1
HBV genotype and geographic distribution

| Genotype | Geographic distribution | Comment |
|---|---|---|
| A | Africa, India, Northern | Precore variant rarely found |
| | Europe, US | |
| B | Asia, US | |
| C | Asia, US | Precore variant found in 50%. |
| | | More severe liver disease than |
| | | with B in some studies |
| D | India, Middle East, | Associated with anti-HBe- |
| | Southern Europe, US | positive chronic HBV infection |
| | | in the Mediterranean region. |
| | | Precore variant found in >70% |
| E | West and South Africa | |
| F | Central and South America | |
| G | Europe, US | |
| H | Central and South America, | |
| | California (US) | |

Source: Natural History and Clinical Consequences of Hepatitis B Virus Infection. C. Pan and J. Zhang, Int. J. Med. Sci, 2005, 2: 36-40.

There are several HBV treatments available, for example interferon (IFN) therapy. The UK National Institute for Health and Care Excellence (NICE) recommends in their draft clinical guidelines that PEGylated-interferon alpha-2a therapy should be offered as a first line treatment in chronic HBV infection. However, only around 30% of those treated will actually respond to IFN therapy, which results in patients suffering from unnecessary side-effects and placing a financial burden on the healthcare system. There is therefore a need for an effective and efficient method of predicting whether a patient will respond to IFN therapy.

### SUMMARY OF THE INVENTION

According to a first aspect of the invention, there is provided the use of CD220 as a biomarker to predict whether a patient infected with Hepatitis B virus (HBV) will respond to interferon (IFN) therapy.

According to a further aspect of the invention, there is provided a method of predicting whether a patient infected with Hepatitis B virus (HBV) will respond to interferon therapy, comprising:

(a) obtaining a test biological sample from the patient;
(b) quantifying the amounts of each of the biomarkers defined herein;
(c) comparing the amounts of each of the biomarkers in the test biological sample with the amounts present in one or more reference samples obtained from one or more responders to interferon therapy and/or one or more reference samples obtained from one or more non-responders to interferon therapy, such that
(i) an equivalent level of each of the biomarkers in the test biological sample compared with the reference sample(s) from responders; or
(ii) a difference in the level of each of the biomarkers in the test biological sample compared with the reference sample(s) from non-responders, is indicative of whether the patient will respond to interferon therapy.

A further aspect of the invention provides ligands, such as naturally occurring or chemically synthesised compounds, capable of specific binding to the biomarkers of the invention. A ligand according to the invention may comprise a peptide, an antibody or a fragment thereof, or an aptamer or oligonucleotide, capable of specific binding to the biomarker. The antibody can be a monoclonal antibody or a fragment thereof capable of specific binding to the biomarker. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or His (e.g. hexa-His) tag.

Also provided by the invention is the use of one or more ligands as described herein, which may be naturally occurring or chemically synthesised, and is suitably a peptide, antibody or fragment thereof, aptamer or oligonucleotide, or any other natural or artificial chemical entity capable of recognizing the biomarkers, or the use of a biosensor of the invention, or an array of the invention, or a kit of the invention to detect and/or quantify the biomarker. In these uses, the detection and/or quantification can be performed on a biological sample such as from the group consisting of whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof.

Kits are provided for performing methods of the invention. Such kits will suitably comprise a ligand according to the invention, for detection and/or quantification of the biomarkers of the invention, and/or a biosensor, and/or an array as described herein, optionally together with instructions for use of the kit.

Therefore, according to a further aspect of the invention, there is provided a kit comprising reagents and/or a biosensor capable of detecting and/or quantifying each of the biomarkers as defined herein, for use in predicting whether a patient infected with Hepatitis B virus (HBV) will respond to interferon therapy.

### BRIEF DESCRIPTION OF THE FIGURES

FIG. 1: Expression of selected biomarkers in IFN responders versus non-responders in HBV e+ patients.

FIG. 2: Training set (50% of samples). Scatter plot of the sCD expression for responders and non-responders of the 50% initial training dataset.

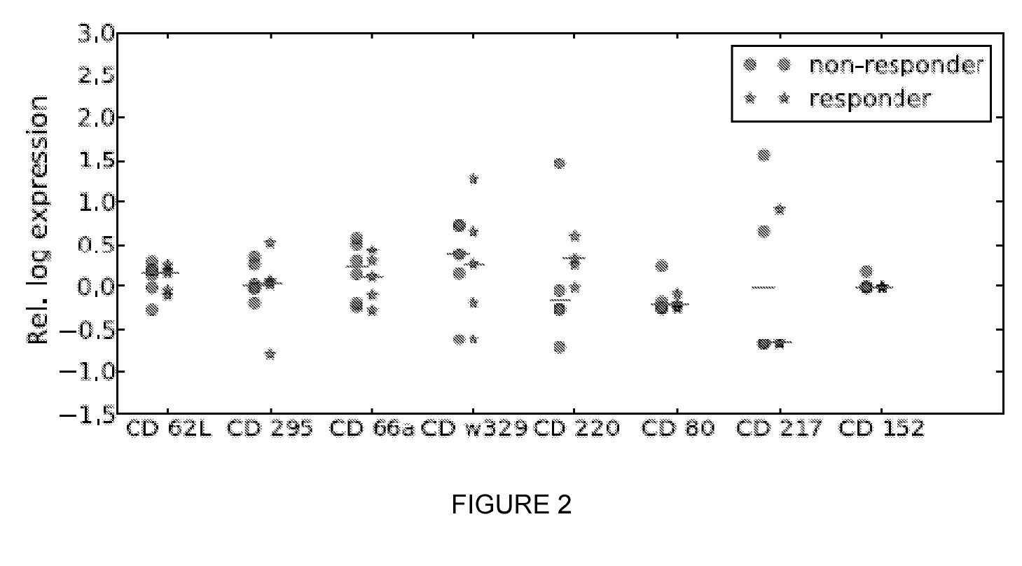

FIG. 3: Training set (full data set). Scatter plot of the sCD expression for responders and non-responders on the full 100% dataset, including training and prediction phase.

### DETAILED DESCRIPTION OF THE INVENTION

According to a first aspect of the invention, there is provided the use of CD220 as a biomarker to predict whether a patient infected with Hepatitis B virus (HBV) will respond to interferon (IFN) therapy.

In one embodiment, the use additionally comprises CD295 as a further biomarker.

In a further embodiment, the use additionally comprises CD62L, CD152, CDw329 and/or CD80 as further biomarkers. In a yet further embodiment, the use additionally comprises CD66a and CD217b/r as further biomarkers.

According to an alternative aspect of the invention, there is provided the use of CD295 as a biomarker to predict whether a patient infected with Hepatitis B virus (HBV) will respond to interferon (IFN) therapy.

According to a further aspect of the invention there is provided the use of CD220 and CD295 as a specific panel of biomarkers to predict whether a patient infected with Hepatitis B virus (HBV) will respond to interferon (IFN) therapy.

The term “biomarker” refers to a distinctive biological or biologically derived indicator of a process, event, or condition. In particular, biomarkers of the invention are used to identify patients which are most likely to respond to a particular therapeutic treatment. Biomarkers can also be used in methods of diagnosis, e.g. clinical screening, and prognosis assessment and in monitoring the results of therapy, drug screening and development. Biomarkers and uses thereof are valuable for identification of new drug treatments and for discovery of new targets for drug treatment. In one embodiment, the biomarkers as defined herein are used in vitro.

References herein to “CD” or “Cluster of Differentiation” refer to cell surface proteins that are present on white blood cells and other tissue types. In the context of the invention, the term “CD” refers to a cell surface leukocyte molecule recognised by a given monoclonal or group of monoclonal antibodies or polyclonal antibodies which specifically ‘cluster’ to the antigen/molecule in question or a polyclonal antibody.

It should be noted that CD220 may also be referred to as “insulin receptor”. CD295 may also be referred to as “leptin receptor”.

A patient will be considered to “respond” to IFN therapy if they have a sustained response after treatment with IFN. Interferon is intended to treat HBV infected patients through two mechanisms: firstly, IFN inhibits synthesis of viral DNA and has a direct antiviral effect; and secondly, IFN directs the patient's immune response to infected hepatocytes by stimulating natural killer and helper T cells (see, Asselah et al. Clinics in Liver Disease, 11 (2007) 839-849). Therefore, IFN therapy results in reduction of HBV replication (indicated by a reduction of HBV DNA in serum).

In particular, response to therapy can be defined as undetectable HBV DNA (for example, less than 10⁵ copies/mL) in serum, sustained loss of HBV “e” Antigen (HBeAg) with or without detection of anti-HBe (HBeAg seroconversion), normalisation of ALT (alanine aminotransferase) and decrease in liver necroinflammation or fibrosis.

Use of the biomarkers defined herein will be able to identify which patients will successfully respond to interferon therapy. This will save the costs of futile IFN therapy in non-responding patients and the associated costs for managing IFN side-effects.

The advantages to patients would result from a personalised treatment approach, better clinical outcome and better compliance. For the medical staff it would be a very useful tool to better tailor treatment to individual patient needs and to help with long term monitoring. For the healthcare system itself there would be significant savings as the expensive IFN treatment would be reserved for the patients that are most likely to have a sustainable response.

In one embodiment, the biomarker is a soluble CD (sCD).

Many, if not all CD molecules produce soluble forms that are released from the cell surface by alternative splicing, proteolytic cleavage, dissociation or other mechanisms. Thus in the context of the invention, the term sCD (i.e. soluble CD molecule) is synonymous with the term secreted or soluble or shed CD (sCD) and refers to a released form of a leukocyte molecule that is typically found expressed at the cell surface and in which at least a portion of that molecule is recognised by a given monoclonal or group of monoclonal antibodies or polyclonal antibody as herein described. It should be noted however, that the antibody used to recognise the CD molecule may not be a naturally occurring monoclonal or polyclonal. It may be engineered, an artificial construct consisting of an expressed fragment derived from an antibody molecule with intact recognition, or it may be a non-protein molecular recognition agent, or a protein recognition agent, which is not an antibody, or is an antibody hybrid, for example made by introducing antibody binding sites into a different scaffolding.

Advantageously, as defined in WO 03/075016, a soluble form of sCD is generated by various mechanisms including, but not limited to, any of those selected from the group consisting of the following: alternative splicing, proteolytic cleavage and dissociation.

It has been shown that patterns of sCDs in blood appear to correlate with the presence of specific diseases. This led to the idea that patterns of sCD antigen biomarkers could be used as the basis of a generalised system for disease diagnosis and monitoring. Many CD antigens play an important role in immune responses and therefore a specific sCD signature may reflect the on-going immune response to a particular disease process. Pattern recognition software may be used to analyse databases of proteomic profiles, and to define disease-specific biomarker signatures. The diagnostic test developed can be easily implemented using standard ELISA technology, which is readily available in all diagnostic laboratories and can use blood (serum or plasma) as the principal diagnostic specimen.

In one embodiment, the interferon (IFN) therapy is alpha interferon therapy, such as the use of alpha-2a or the use of alpha-2b interferon. In a further embodiment, the alpha interferon therapy comprises the use of pegylated alpha interferon. In a yet further embodiment, the interferon (IFN) therapy comprises the use of PEGylated interferon alpha-2a. In an alternative embodiment, the IFN therapy comprises the use of PEGylated interferon alpha-2b.

In a further alternative embodiment, the alpha interferon therapy comprises the use of non-pegylated alpha interferon.

The draft NICE clinical guidance “Diagnosis and management of chronic hepatitis B in children, young people and adults” has recently been published and the final guideline is expected mid-2013. The importance of chronic HBV and health cost is emphasised by this recent release and the draft recommendations.

The current HBV treatment options of IFN (PEGylated interferon alpha-2a) or nucleoside analogues (e.g. entecavir, tenofovir etc.) have several drawbacks: low effectiveness, cost and side effects (especially IFN) and high viral resistance (to nucleoside analogues). The current estimates are that within a cohort of chronic HBV patients seen in an out-patients clinic in a low endemic country such as the UK, approximately 20% of patients are HBeAg-positive and being treated. 40-50% of patients who are HBeAg positive are in the immune tolerant phase (e+ inactive) and therefore monitored.

The ideal approach to antiviral therapy in chronic HBV remains uncertain. Conventional IFNα, with its dual immunomodulatory and antiviral effects, seems to have an improved disease outcome and complication-free survival but only in a small proportion of patients who are sustained responders. Newer PEGylated IFN alpha-2a (Pegasys®) is licensed globally for the treatment of chronic HBV while PEGylated IFN alpha-2b (PegIntron®) is licensed only in specific high endemic countries (but used off-label in others such as US).

There are now five nucleoside/nucleotide analogues approved for the treatment of chronic HBV, namely lamivudine (Epivir-HBV), adefovir (Hepsera), entecavir (Baraclude), telbivudine (Tyzeka), and tenofovir (Viread). Antiviral therapy may require indefinite treatment and often starts at a young age. There are therefore issues with patient compliance which can lead to viral resistance.

HBV has a reported mutation rate of 10 times greater compared with other DNA viruses and some mutations are due to selective pressure from antiviral therapy. There are five clinically relevant HBV types: wild-type HBV, precore mutants, core promoter mutants, tyrosine-methionine-aspartate-aspartate (YMDD) mutants induced by lamivudine treatment, and asparagine to threonine (rtN236T) mutants recently identified in patients with adefovir treatment. Increase in concentrations of precore mutation in proportion to wild-type HBV is associated with flare-ups in chronic HBV.

There is therefore a need to prevent over-use of these oral antiviral (OAV) therapies, to try and reduce the emergence of viral resistance. The biomarkers defined herein will help to promote IFN therapy as a first line treatment by positively identifying patients who will successfully respond to treatment and therefore reduce the use of OAVs.

Furthermore, the drug costs differ substantially; the long duration of therapy with OAVs clearly leads to significantly greater expenditure over the long term when compared to the shorter course of therapy with IFN.

However, given the perceived frequency and severity of side effects, it is clear that clinical practice currently tends towards OAV treatment even though the cost to the healthcare system and the risk of developing viral resistance is higher. Therefore, the biomarkers presented herein provide a way of identifying patients that will benefit from short course therapy with IFN which has advantages including a lack of drug resistance, a finite and defined treatment course, and a higher likelihood for Hepatitis B surface antigen (HBsAg) clearance (see Perillo R. Hepatology, 2009, 49(5 Suppl): S103-11).

In one embodiment, the patient infected with Hepatitis B virus (HBV) is chronically infected.

References herein to “chronic infection” refer to individuals which have been infected for a long period of time (for example, for longer than 3 months, 6 months, 9 months or over 12 months). Chronically infected HBV patients have detectable levels of HBV DNA and Hepatitis B surface antigen (HBsAg) and persistently elevated levels of ALT and chronic liver inflammation which can lead to cirrhosis.

HBV infection is divided into four distinct stages: the immune tolerant phase, the immune clearance phase, and the inactive carrier phase with or without reactivation. The clinical pathology of chronic HBV is brought about mechanistically by the body's immune system reacting to the presence of the virus (so-called necroinflammatory disease). It was originally speculated that some chronic HBV patients were immunotolerant and that treatment of those patients was unnecessary because there was no likelihood of disease. In the immune clearance phase (HBV “e” Antigen positive) liver damage occurs and all patients are offered treatment.

Neonatal and childhood infection has a very low rate of spontaneous clearance and this sub-group of patients, in the UK represented primarily by Asian minorities, suffers disproportionately from chronic HBV infection. A recent paper by Patrick Kennedy et al. (Gastroenterology, 2012, 143: 637-645) and an editorial postulate in the same issue states that young chronic HBV patients who are most likely to go chronic but are least likely to be treated may now be considered more suitable for treatment after demonstrating that there is evidence of chronic liver damage not diagnosed on current monitoring protocols. Currently these patients, with mild chronic HBV, are not offered treatment as treatment-maintained response can be achieved with long-term nucleoside/nucleotide analogue therapy (i.e. OAVs). However, given the emergence of resistance there is a tendency to delay treatment until there is evidence of liver damage.

On the other hand sustained response can be achieved with IFN-based therapy with its dual immunomodulatory and antiviral effects, but only in a small proportion of patients who are sustained responders. Therefore, the biomarkers presented herein would be able to identify young patients with early stage/mild chronic HBV that would benefit from IFN therapy before suffering from liver damage.

In one embodiment, the patient infected with Hepatitis B virus (HBV) is HBV “e” antigen (HBeAg) positive (e+), i.e. the patient is in the immune clearance phase. In a further embodiment, the patient is HBeAg positive active (e+ active).

In an alternative embodiment, the patient infected with Hepatitis B virus (HBV) is HBV “e” antigen (HBeAg) negative. In a further alternative embodiment, the patient infected with Hepatitis B virus (HBV) is HBV surface antigen (HBsAg) positive. In a yet further alternative embodiment, the patient infected with Hepatitis B virus (HBV) is HBV surface antigen (HBsAg) negative.

HBV infection is divided into four distinct stages: the immune tolerant phase, the immune clearance phase, and the inactive carrier phase with or without reactivation. The key parameters of each stage are summarized in Table 2. It should be noted that rare cases might not follow the exact pattern described below.

TABLE 2
Summary of the stages of HBV infection and unmet clinical need

| | | | Inactive | |
|---|---|---|---|---|
| | | | carrier (low | Inactive carrier |
| | Immune | Immune | or non- | reactivation |
| Measure | tolerant | clearance | replicative) | (immunoescape) |
| Hepatitis B | Positive | Positive | Positive, | Positive |
| surface | | | becomes | |
| antigen | | | negative in | |
| (HBsAg) | | | resolution | |
| Hepatitis B | Positive | Positive | Negative | Negative |
| extracellular | | | | |
| “e” antigen | | | | |
| (HBeAg) | | | | |
| HBV DNA | High | Falling | Low or not | High |
| | | | detectable | |
| ALT | Normal | Elevated | Usually | Elevated |
| | | | normal | |
| Anti-HBe | Negative | Negative | Sometimes | Sometimes |
| (HBeAb) | | | | |
| Anti-HBs | Sometimes | | | |
| (HBsAb) | (resolution) | | | |

The current consensus chronic HBV management recommendation, confirmed by NICE guidance, is to offer treatment to chronic HBeAg-positive or HBeAg-negative Hepatitis B patients. However, currently only a minority of these HBeAg positive patients are offered treatment in the UK once they show signs of liver damage. The biomarkers, methods and kits defined herein will help promote the use of IFN therapy by offering a simple and effective way of positively identifying patients who will benefit from treatment.

According to a further aspect of the invention, there is provided a method of predicting whether a patient infected with Hepatitis B virus (HBV) will respond to interferon therapy, comprising:

(a) obtaining a test biological sample from the patient;
(b) quantifying the amounts of each of the biomarkers defined herein;
(c) comparing the amounts of each of the biomarkers in the test biological sample with the amounts present in one or more reference samples obtained from one or more responders to interferon therapy and/or one or more reference samples obtained from one or more non-responders to interferon therapy, such that
(i) an equivalent level of each of the biomarkers in the test biological sample compared with the reference sample(s) from responders; or
(ii) a difference in the level of each of the biomarkers in the test biological sample compared with the reference sample(s) from non-responders, is indicative of whether the patient will respond to interferon therapy.

It should be noted that references to a biomarker amounts or levels also include references to a biomarker range.

Using the data obtained in the Examples described herein (excluding an outlying sample), the biomarker ranges (i.e. “normal ranges”) for CD220 were as follows:
Responders: 5.8-10.9 ng/ml
Non-responders: 1.4-5.7 ng/ml

These ranges can therefore be used to predict whether an individual will respond to interferon therapy. In one embodiment, if a test biological sample (e.g. a serum sample) for a patient infected with HBV contains greater than 5.8 ng/ml, such as 5.8-10.7 ng/ml, of CD220 then this indicates that the patient will respond to interferon therapy.

In one embodiment, the method is conducted on samples taken on two or more occasions from a test subject.

In one embodiment, the method further comprises detecting a change in the amount of the biomarkers in samples taken when the patient infected with Hepatitis B virus (HBV) is chronically infected.

References herein to “responder” refer to an individual infected with HBV who has demonstrated a response to interferon therapy. References herein to “non-responder” refer to an individual infected with HBV who has not demonstrated a response to interferon therapy.

Individuals that do not respond to IFN therapy (“non-responders”) are defined as those who do not have a sustained response after treatment with IFN. Individuals who respond to IFN therapy (“responders”) have a clinically relevant response that allows discontinuation of therapy. Currently, this is typically defined as the loss of HBeAg and the development of HBeAb, and/or a reduction in liver inflammation (normal liver function tests) with a permanent reduction in HBV DNA to a level not requiring antiviral therapy (currently 2000 IU/ml), and/or the loss of HBsAg.

The present inventors have made the surprising discovery that CD220 levels are elevated in HBV infected individuals that respond to interferon therapy. Therefore, in a further embodiment, a higher amount (i.e. level) of CD220 in the test biological sample compared with the sample from an individual that does not respond to interferon therapy is indicative that the patient infected with Hepatitis B virus (HBV) will respond to interferon therapy.

In one embodiment, the higher level is a >1 fold difference relative to the reference sample, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15, 20, 25, 30, 35, 40, 45, 50, 55, 60, 65, 70, 75, 80, 85, 90, 95, 100 or any ranges there between. In one embodiment, the higher level is between 1 and 75 fold difference relative to the reference sample, such as between 1.5 and 10, in particular between 1.5 and 5.

In one embodiment, the equivalent level is the same or a similar level of CD220 in the test biological sample compared with the sample from an individual that does respond to interferon therapy.

References herein to the “same” level of biomarker indicate that the level of biomarker measured in each sample is identical (i.e. when compared to the selected reference). References herein to a “similar” level of biomarker indicate that levels are not identical but the difference between them is not statistically significant (i.e. the levels have comparable quantities).

In one embodiment, one or more of the biomarkers may be replaced by a molecule, or a measurable fragment of the molecule, found upstream or downstream of the biomarker in a biological pathway.

In one embodiment, the method additionally comprises the use of a statistical algorithm to determine the likelihood of whether a patient will respond to interferon therapy.

It will be appreciated that the skilled person would be able to prepare a statistical algorithm based on the serum CD220 levels obtained from responders and non-responders. For example, in the Examples described herein, the statistical algorithm was generated by using serum samples obtained from responders and non-responders to establish a threshold value based on CD220 expression. The threshold value was determined using the midpoint between the median of both sample groups.

In one embodiment, the method is performed with a positive predictive value (PPV) of at least 50%, for example at least 55%, 60%, 65%, 70%, 75%, 80% or 85%, in particular at least 90%, such as at least 95% or 99%.

The invention disclosed herein has been shown to operate with a significantly high accuracy of 90% PPV. Reference herein to the “positive predictive value” is a term well known in the art and refers to the proportion of positive test results that are true positives.

The term “detecting” as used herein means confirming the presence of the biomarkers of the invention present in the sample. Quantifying the amount of the biomarkers present in a sample may include predicting the concentration of the biomarkers present in the sample. Detecting and/or quantifying may be performed directly on the sample, or indirectly on an extract therefrom, or on a dilution thereof.

In alternative aspects of the invention, the presence of the biomarkers of the present invention are assessed by detecting and/or quantifying antibody or fragments thereof capable of specific binding to the biomarkers that are generated by the subject's body in response to the biomarkers and thus are present in a biological sample from a subject having HBV.

Detection and/or quantification of biomarkers of the invention may be performed by detection of the biomarkers or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

In one embodiment, the quantifying is performed by measuring the concentration of the biomarkers in each sample.

In one embodiment, the detecting and/or quantifying is performed using an immunological method.

In one embodiment, the detecting and/or quantifying is performed using a biosensor or a microanalytical, microengineered, microseparation or immunochromatography system.

The biomarkers described herein may be directly detected, e.g. by SELDI or MALDI-TOF. Alternatively, the biomarkers may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing and/or monitoring according to the invention may comprise analysing a plasma, serum or whole blood sample by a sandwich immunoassay to detect the presence or level of the biomarkers described herein. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the biomarkers of the invention may be performed using an immunological method, involving an antibody, or a fragment thereof capable of specific binding to the biomarker. Suitable immunological methods include sandwich immunoassays, such as sandwich ELISA, in which the detection of the biomarkers is performed using two antibodies which recognize different epitopes on a biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes, which are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc.) is labelled with biotin and the other partner (surface, e.g. well, bead, sensor etc.) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for a biomarker of the invention may be immobilised on an avidin or streptavidin surface, the immobilised ligand may then be exposed to a sample containing or suspected of containing the biomarker in order to detect and/or quantify a biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, fragments (such as FAb, F(Ab′)2, Fv, disulphide linked Fv, scFv, diabody), fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, monoclonal antibodies or engineered antibodies, including phage antibodies raised against the sCD or their membrane bound form are used for their detection. However, non-protein agents may also in principle be used to detect sCDs. Similarly the detecting molecule may contain antibody binding site fragments incorporated into the scaffold of another molecule or an engineered scaffold. Commercially available kits for measuring CD levels include those from Diaclone 1, Bd A Fleming BP 1985 F-25020 Besancon Cedex-France and Medsystems Diagnostics GmbH, Rennweg 95b, A-1030 Vienna Austria.

Suitable techniques for measuring sCDs include but are not limited to immunoassays including ELISA using commercially available kits such as those described above, flow cytometry particularly multiplexed particle flow cytometry as herein described. Those skilled in the art will be aware of other suitable techniques for measuring CD levels in samples from an individual including antibody ‘chip’ array type technologies or chip technologies utilizing non-classical antibody binding site grafted molecules.

In one particular embodiment, the method of measuring sCDs comprises an MSD® assay as defined hereinbefore with the modifications described herein.

Methods involving detection and/or quantification of one or more biomarkers of the invention can be performed on bench-top instruments, or can be incorporated onto disposable, diagnostic or monitoring platforms that can be used in a non-laboratory environment, e.g. in the physician's office or at the patient's bedside.

Methods of the invention may involve simple lateral flow analysis in order to detect the biomarkers described herein. Lateral flow assays have the advantage of combining various reagents and process steps all in one assay. These types of assays are used to detect an analyte in a fluid sample. The fluid moves from one end of the assay to the other, mainly by capillary action, passing through various “mobile” or “capture” reagents that are used to detect the analyte. If the analyte is present, a detectable signal is produced when the fluid sample reaches the opposite end of the assay from where it was administered.

As used herein, the term “biosensor” means anything capable of detecting the presence of the biomarker. Examples of biosensors are described herein.

Biosensors according to the invention may comprise a ligand or ligands, as described herein, capable of specific binding to the biomarkers. Such biosensors are useful in detecting and/or quantifying a biomarker of the invention.

The identification of key biomarkers specific to a disease is central to integration of diagnostic procedures and therapeutic regimes. Using predictive biomarkers appropriate diagnostic tools such as biosensors can be developed; accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

The biomarkers of the invention can be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with the biomarker. On exposure, the biomarker reacts with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several substances. Reversible and irreversible sensors can be designed to meet different requirements, and continuous monitoring of a particular biomarker of interest is feasible.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Suitable biosensors for performing methods of the invention include “credit” cards with optical or acoustic readers. Biosensors can be configured to allow the data collected to be electronically transmitted to the physician for interpretation and thus can form the basis for e-neuromedicine.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

High-throughput screening technologies based on the biomarkers, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format.

Detecting and/or quantifying can be performed by any method suitable to identify the presence and/or amount of a specific biomarker in a biological sample from a patient or a purification or extract of a biological sample or a dilution thereof. In methods of the invention, quantifying may be performed by measuring the concentration of the biomarker in the sample or samples. Biological samples that may be tested in a method of the invention include whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner. In one embodiment, the biological sample comprises whole blood, serum or plasma. In a further embodiment, the biological sample comprises serum, such as non-activated or unstimulated serum. In a further alternative embodiment, the biological sample comprises plasma.

In one embodiment, the method additionally comprises administering interferon to a patient predicted to respond to interferon therapy.

Thus, according to a further aspect of the invention, there is provided a method of treatment of a patient infected with Hepatitis B virus (HBV), wherein said method comprises the steps of:

(a) obtaining a test biological sample from the patient;
(b) quantifying the amounts of each of the biomarkers defined herein;
(c) comparing the amounts of each of the biomarkers in the test biological sample with the amounts present in one or more reference samples obtained from one or more responders to interferon therapy and/or one or more reference samples obtained from one or more non-responders to interferon therapy, such that
(i) an equivalent level of each of the biomarkers in the test biological sample compared with the reference sample(s) from responders; or
(ii) a difference in the level of each of the biomarkers in the test biological sample compared with the reference sample(s) from non-responders, is indicative of whether the patient will respond to interferon therapy; and
(d) administering interferon to a patient predicted to respond to interferon therapy.

It will be appreciated that each of the embodiments for the method of predicting aspect of the invention apply equally to this method of treatment aspect of the invention.

According to a further aspect of the invention, there is provided a kit comprising reagents and/or a biosensor capable of detecting and/or quantifying each of the biomarkers as defined herein, for use in predicting whether a patient infected with Hepatitis B virus (HBV) will respond to interferon therapy.

In one embodiment, the reagents comprise one or more components for conducting an ELISA.

In one embodiment, the kit additionally comprises a vial of interferon. This may then be administered to the patient if they are indicated to be a responder to interferon therapy.

Suitably a kit according to the invention may contain one or more components selected from the group: a ligand specific for the biomarker or a structural/shape mimic of the biomarker, one or more reference values or ranges, one or more reagents and one or more consumables; optionally together with instructions for use of the kit in accordance with any of the methods defined herein.

The following studies illustrate the invention.

### Example 1

### Preliminary Proof of Concept Study

The samples for the proof of concept study were provided from the retained samples collected in the outpatient clinic located at the Royal London Hospital. All the samples were analysed for 66 unique sCD specificities. The samples analysed were: 15 controls and 46 HBV patients.

Anonymised clinical data on the patients was obtained, including treatments given and clinical response. The current UK clinical practice is that all HBeAg positive (e+) active patients are offered treatment of either IFN or oral anti-viral therapy and patient choice is the final determinant of therapy choice (rather than potential response to treatment). Based on clinical response to treatment, some additional analysis was performed. The results are based on a very small sample of e+ active patients (n=10), of which only 5 were offered IFN therapy.

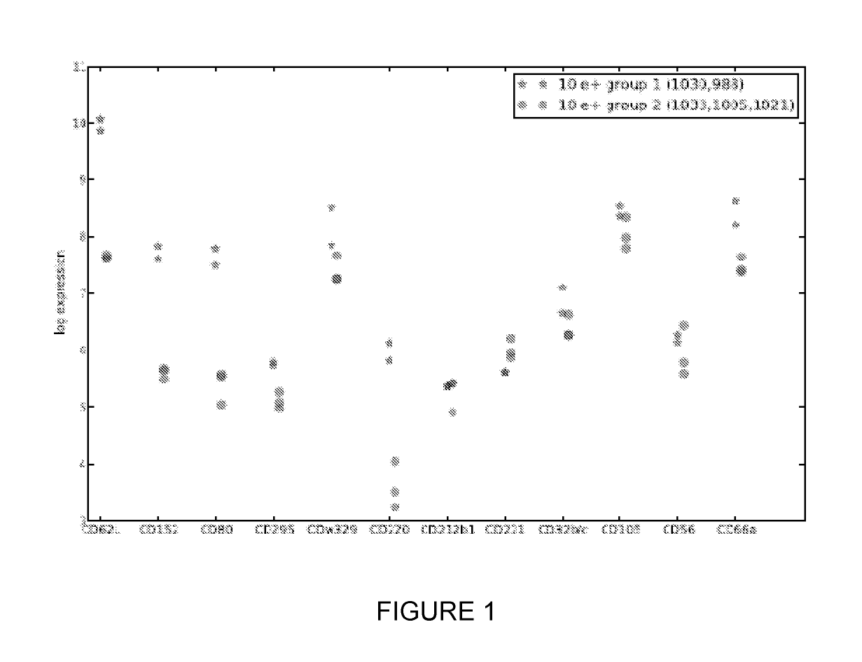

Bearing in mind the very small sample size, the results were very clearly discriminative between interferon responders and non-responders as shown in FIG. 1. The top discriminative specificities were CD 62L, CD 152, CD w329, CD 80, CD 220 and CD 295.

### Example 2

### Design of a Preliminary Validation Study

Further samples from the outpatient clinic were provided to validate the data obtained in Example 1. The aim was to confirm the ability to predict response to IFN in HBeAg positive patients. Eight sCD biomarkers (sCD62L, sCD295, sCD66a, sCDw329, sCD220, sCD80, sCD217b/r, sCD152) that could be deployed on the more reliable and quantitative Meso Scale Diagnostic Immunoassay platform were selected.

32 samples were obtained from the outpatient clinic as follows:
12 non-responders;
10 responders; and
10 HBeAg positive patients with normal liver function and not yet treated.

The responder and non-responder samples were heterogeneous so that the confounding effect could be excluded (for example a biomarker that could pick only Chinese patients). All the patients were naïve and they had been treated with PEGylated IFN for one year.

TABLE 3
Patient description responders (R) versus non responders (NR)

| | R | NR |
|---|---|---|
| Genotype A | 3 (British, Chinese, | 3 (2 British, 1 Bangladeshi) |
| | Bangladeshi) | |
| Genotype B | 2 (2 Chinese) | 0 |
| Genotype C | 3 (1 British, 2 Bangladeshi) | 5 (2 Chinese, 1 Thai, 1 |
| | | British, 1 Afro-Caribbean) |
| Genotype D | 2 (1 Pakistani, 1 Somalian) | 3 (2 Eastern European, 1 |
| | | Bangladeshi) |
| Genotype E | 0 | 1 (Afro-Caribbean) |

The raw data was analysed in 2 stages using proprietary algorithms for pattern extraction, normalisation and classification:
Training set (50% of samples unblinded)
Prediction of the remaining 50% of samples (the clinical data were not made available to the bioinformatician at this stage)

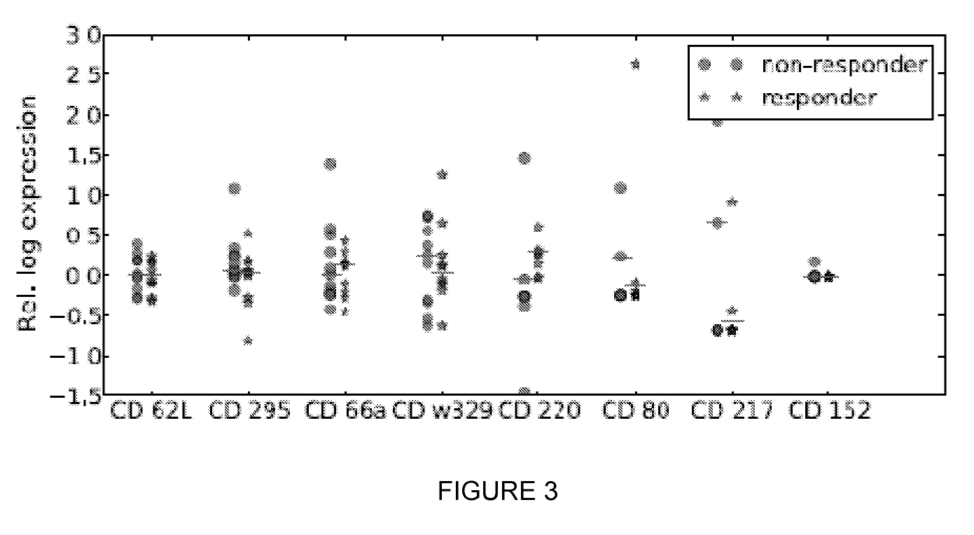

A basic linear predictor was considered to identify a decision boundary/threshold value between non-responders and responders using the antigen sCD220. The threshold value was determined using the midpoint between the median of both sample groups. Using this decision function to predict the disease status of the remaining 50% of samples (test samples), they could be classified with 100% accuracy (see also FIG. 3).

On the training dataset, one outlier was identified (sCD220, highest relative expression level). Possible reasons for this outlying sample were investigated, but no conclusive evidence could be found. The only difference identified is a very low pre-treatment viral load and the clinical decision to treat was based on supposed liver function deterioration.

When considering other training/test splits between the total set of 32 samples, the outlying sample did reduce the specificity when being part of the test set. However, the accuracy never dropped below 97%, which corresponds to a single mis-classification caused by the outlying sample.

The impact of genotype and ethnic origin as possible confounding factors were also investigated. Reassuringly, none of the considered biomarkers was significantly affected by any of these factors.

Liver function was also looked at, and in particular ALT level. Confounding due to ALT levels was checked but it is not likely to be the driving factor (i.e. ALT as a predictor does not have the specificity and sensitivity of sCD220).

The conclusions of Example 2 are therefore as follows:

1. Initial signatures are in part validated although the ranking of discrimination has changed
2. sCD220 could be used to predict out of sample with between 97%-100% accuracy, when splitting the samples into equal sized training/test sets.

TABLE 4
P-values of differential expression for the considered antigens.

| | | | | | | sCD217 | |
|---|---|---|---|---|---|---|---|
| sCD62L | sCD295 | sCD66a | sCDw329 | sCD220 | sCD80 | b/r | sCD152 |
| 0.67 | 0.164 | 0.512 | 0.92 | 0.06 | 0.515 | 0.33 | 0.5 |

In the initial screening experiment, a second biomarker (sCD295) was identified that seems a plausible candidate for differentiation. Addition of this biomarker to sCD220 would help to increase the accuracy and sensitivity of this test even further.

The ability to predict the response to IFN has attracted much research for reliable and clinically acceptable biomarkers. It is clear that the manufacturers of IFN have not thus far been successful.

The biomarkers identified herein (especially sCD220) are surprising because they are not the obvious candidates for prediction based on current scientific knowledge of immune pathways.

### REFERENCES

Natural History and Clinical Consequences of Hepatitis B Virus Infection. C. Pan and J. Zhang, International Journal of Medical Sciences, 2005, 2: 36-40
Hepatitis B virus epidemiology, disease burden, treatment, and current and emerging prevention and control measures. D. Lavanchy (WHO), Journal of Viral Hepatitis, 2004, 11: 97-107
Diagnosis and treatment of chronic hepatitis B. R. D'Souza and G. Foster, Journal of Royal Society of Medicine, 2004, 97:318-321
The immune response during hepatitis B virus infection. A. Bertoletti and A. Gehring, Journal of General Virology, 2006, 87: 1439-1449
Hepatitis B, World Health Organisation, WHO/CDS/CSR/LYO 2002.2
American Association for the Study of Liver Diseases (AASLD) practice guidelines for Chronic Hepatitis B. A. Lok and B. McMahon, 2003
Early on-treatment prediction of response to peginterferon alfa-2a for HBeAg-negative chronic hepatitis B using HBsAg and HBV DNA levels. Rijckborst V et al. Hepatology, 2010, 52(2):454-61.

Early serum HBsAg drop: a strong predictor of sustained virological response to pegylated interferon alfa-2a in HBeAg-negative patients. Moucari R et al. Hepatology, 2009, 49(4):1151-7.
Alpha-1 antitrypsin is a potential biomarker for hepatitis B. Tan et al. Virology Journal, 2011, 8:274
Effect of lamivudine treatment on plasma levels of transforming growth factor β1, tissue inhibitor of metalloproteinases-1 and metalloproteinase-1 in patients with chronic hepatitis B. Flisiak R et al. World Journal of Gastroenterology 2004, 10(18):2661-2665

Predictive value of HBsAg quantification for determining the clinical course of genotype C HBeAg-negative carriers. Park, H et al. Liver International, 2011.

1. Use of CD220 as a biomarker to predict whether a patient infected with Hepatitis B virus (HBV) will respond to interferon (IFN) therapy.

2. Use according to claim 1, which comprises CD295 as a further biomarker.

3. Use according to claim 1 or claim 2, which additionally comprises CD62L, CD152, CDw329 and/or CD80 as further biomarkers.

4. Use according to any one of claims 1 to 3, which additionally comprises CD66a and CD217b/r as further biomarkers.

5. Use of CD220 and CD295 as a specific panel of biomarkers to predict whether a patient infected with Hepatitis B virus (HBV) will respond to interferon (IFN) therapy.

6. Use according to any one of claims 1 to 5, wherein the biomarker is a soluble CD (sCD).

7. Use according to any one of claims 1 to 6, wherein the interferon (IFN) therapy is alpha interferon therapy.

8. Use according to claim 7, wherein the alpha interferon therapy comprises the use of interferon alpha-2a.

9. Use according to claim 7, wherein the alpha interferon therapy comprises the use of interferon alpha-2b.

10. Use according to any one of claims 7 to 9, wherein the alpha interferon therapy comprises the use of pegylated alpha interferon.

11. Use according to any one of claims 7 to 9, wherein the alpha interferon therapy comprises the use of non-pegylated alpha interferon.

12. Use according to any one of claims 1 to 11, wherein the patient infected with Hepatitis B virus (HBV) is chronically infected.

13. Use according to any one of claims 1 to 12, wherein the patient infected with Hepatitis B virus (HBV) is HBV e antigen (HBeAg) positive (e+).

14. A method of predicting whether a patient infected with Hepatitis B virus (HBV) will respond to interferon therapy, comprising:
(a) obtaining a test biological sample from the patient;
(b) quantifying the amounts of each of the biomarkers defined in any one of claims 1 to 5;
(c) comparing the amounts of each of the biomarkers in the test biological sample with the amounts present in one or more reference samples obtained from one or more responders to interferon therapy and/or one or more reference samples obtained from one or more non-responders to interferon therapy, such that
(i) an equivalent level of each of the biomarkers in the test biological sample compared with the reference sample(s) from responders; or
(ii) a difference in the level of each of the biomarkers in the test biological sample compared with the reference sample(s) from non-responders, is indicative of whether the patient will respond to interferon therapy.

15. A method as defined in claim 14, wherein a higher level of CD220 in the test biological sample compared with the one or more reference samples obtained from one or more non-responders to interferon therapy is indicative that the patient infected with Hepatitis B virus (HBV) will respond to interferon therapy.

16. A method as defined in claim 14 or claim 15, which additionally comprises the use of a statistical algorithm to determine the likelihood of whether a patient will respond to interferon therapy.

17. A method as defined in any one of claims 14 to 16, wherein the method is performed with a positive predictive value (PPV) of at least 50%, such as at least 75%, in particular at least 90%.

18. A method as defined in any one of claims 14 to 17, wherein quantifying is performed by measuring the concentration of the biomarkers in each sample.

19. A method as defined in any one of claims 14 to 18, wherein detecting and/or quantifying is performed using an immunological method.

20. A method as defined in any one of claims 14 to 19, wherein the detecting and/or quantifying is performed using a biosensor or a microanalytical, microengineered, microseparation or immunochromatography system.

21. A method as defined in any one of claims 14 to 20, wherein the biological sample is whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof.

22. A method as defined in claim 21, wherein the biological sample is whole blood, serum or plasma, such as serum.

23. A method as defined in any one of claims 14 to 22, which additionally comprises administering interferon to a patient predicted to respond to interferon therapy.

24. Use of a kit for predicting whether a patient infected with Hepatitis B virus (HBV) will respond to interferon therapy, wherein said kit comprises reagents and/or a biosensor capable of detecting and/or quantifying each of the biomarkers as defined in any one of claims 1 to 5.

25. The use as defined in claim 24, wherein the reagents comprise one or more components for conducting an ELISA.

26. The use as defined in claim 24 or claim 25, wherein the kit additionally comprises a vial of interferon.
